(19) United States
(12) Patent Application Publication
(10) Pub. No.: US 2026/0159390 A1
(43) Pub. Date: Jun. 11, 2026

(54) SYSTEMS, METHODS, AND APPARATUS FOR PRODUCING NITRIC OXIDE

(71) Applicant: VERO Biotech Inc., Atlanta, GA (US)

(72) Inventors: Matelyn Riordan, Atlanta, GA (US); Christopher Varga, Atlanta, GA (US); Mary Virginia Smith, Atlanta, GA (US)

(21) Appl. No.: 19/097,173

(22) Filed: Apr. 1, 2025

Related U.S. Application Data
(60) Provisional application No. 63/728,392, filed on Dec. 5, 2024.

(51) Int. Cl. C01B 21/24; A61M 16/10; B01J 8/02

(52) U.S. Cl. CPC C01B 21/24; A61M 16/10; B01J 8/0278; A61M 2202/0275

(57) ABSTRACT

Disclosed herein is a device comprising: a media configured to convert nitrogen dioxide into nitric oxide, wherein the media comprises: (a) a support material; and (b) an antioxidant material, wherein at least 15 wt % of the antioxidant material is present in a solid form.

### CROSS-REFERENCE TO RELATED APPLICATIONS

This application claims the benefit of U.S. Provisional Application No. 63/728,392, filed on Dec. 5, 2024, the contents of which are incorporated herein by reference in their entirety.

### FIELD

Some aspects described herein relate to a medical device and, more particularly, to systems and methods for producing and delivering a gas that includes nitric oxide.

### BACKGROUND

Some aspects described herein relate to the production of nitric oxide (NO), which is then typically delivered to a patient in a medical setting.

Nitric oxide is a vasodilator indicated to improve oxygenation and reduce the need for extracorporeal membrane oxygenation, particularly in term and near-term neonates with hypoxic respiratory failure associated with clinical or echocardiographic evidence of pulmonary hypertension in conjunction with ventilatory support. Low concentrations of inhaled nitric oxide can also prevent, reverse, or limit the progression of disorders, which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, hyaline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism, and idiopathic or primary pulmonary hypertension or chronic hypoxia.

Inhaled nitric oxide therapy typically involves the delivery of nitric oxide in parts per billion to parts per million concentrations within a breathing gas, generally composing air or oxygen-enriched air. This breathing gas may contain other components, such as anesthetic agents, nebulized liquids, or other gaseous components, and it is typically conveyed to a patient using either a mechanical or manual ventilation device. In some inhaled nitric oxide delivery systems, the nitric oxide is provided within pressurized tanks, whereas in other systems, the nitric oxide may be generated on demand within the delivery system itself. One such system is described in U.S. Pat. No. 11,744,978, the content of which is incorporated herein in its entirety. In this approach, nitric oxide is produced by a chemical reaction between NO₂ gas and an antioxidant wherein the NO₂ gas is made through a phase-change of liquid N₂O₄. In such systems, the liquid N₂O₄ is typically housed in a pressure vessel with components required for reaction control (e.g., heating and cooling components), reactant mixing, and measurement co-located with the reactants themselves.

Historically, the antioxidant cartridge such as that described in US 20220008679 utilizes an aqueous phase reaction on the surface of the silica gel. The silica gel is coated with a film of antioxidant solution in which the chemical reaction to produce nitric oxide occurs. As NO₂ gas passes through the reaction media, NO₂ diffuses into the aqueous layer, where it can react to form NO. However, another aqueous phase reaction of nitrogen dioxide and water can also occur, for example, it can form nitric acid, reducing the total capacity to produce nitric oxide in the silica gel-based media. Further challenges with derivatized silica gel-based media include antioxidant degradation, which is exacerbated in the presence of water. The degradation reduces the overall conversion potential throughout the cartridge shelf life. Additionally, the use of silica gel poses several challenges for manufacturing. Variability in the particle size distribution of the silica gel can cause differences in cartridge packing, which impacts the flow path through the cartridge. Artifacts such as preferential pathing can cause swings in the NO output from the cartridge reactor, which is nonideal for patients. Therefore, there is a need for a high-efficiency reaction media that can convert NO₂ to NO at higher concentrations with reduced dose output variability throughout its shelf life.

This need and all other needs are at least partially addressed by this disclosure.

### SUMMARY

The present disclosure is directed to a device comprising: a media configured to convert nitrogen dioxide into nitric oxide, wherein the media comprises: (a) a support material; and (b) an antioxidant material, wherein at least 15 wt % of the antioxidant material is present in a solid form.

In still further aspects, the antioxidant material present in the solid form has a particle size of 1 to 1000 microns.

In still further aspects, the antioxidant material comprises ascorbic acid; alpha-, beta-, gamma-, or delta tocopherol; alpha-, beta-, gamma-, or delta-tocotrienol; polyphenols; beta-carotene; or a combination thereof.

In still further aspects, the support material can comprise a molecular sieve, a metal-organic framework (MOF), natural zeolites, synthetic zeolites, polymers of intrinsic microporosity (PIMs), hyper-crosslinked microporous polymers (HCPs), covalent organic frameworks (COFs), conjugated microporous polymers (CMPs), porous aromatic frameworks (PAFs), porous organic cages (PCs), silica gel, or any combination thereof.

Still further disclosed herein is a system for forming nitric oxide comprising: a source of NO₂; a vessel comprising the media of any of one of the examples herein; a patient interface coupled to the vessel and configured to deliver the nitric oxide to a patient.

Also disclosed herein are methods of making the media of any of the examples herein, wherein the method comprises dry blending of the antioxidant material present in the solid form with the support.

Also disclosed are methods of making the media of any of the examples herein, wherein the method comprises mixing a saturated or supersaturated solution of the antioxidant in the solvent with the support material; and removing the solvent to form the antioxidant material present in the solid form

Additional advantages will be set forth in part in the description that follows, and in part will be evident from the description or can be learned by practice of the aspects described below. The advantages described below will be realized and attained by means of the chemical compositions, methods, and combinations thereof, particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate several aspects described below.

FIG. 1 depicts an exemplary schematic of forming NO in a device where a support material is coated with a liquid antioxidant.

FIGS. 2A-2C depict an SEM of an exemplary solid antioxidant material (FIG. 2A), an SEM of an exemplary support material (FIG. 2B), and a schematic of blending two together (FIG. 2C).

FIG. 3 depicts a schematic for an antioxidant media production involving the blending of derivatized silica without antioxidants with solid antioxidant particles according to one aspect.

FIG. 4 depicts a schematic for an antioxidant media production involving the blending of derivatized silica with an antioxidant with solid antioxidant particles according to one aspect.

FIG. 5 depicts a schematic for an antioxidant media production involving the blending of silica gel without antioxidants with solid antioxidant particles according to one aspect.

FIG. 6 depicts a schematic for producing antioxidant media by mixing the saturated antioxidant solution with silica gel and subsequently drying/solvent removal to produce solid antioxidants, according to one aspect.

FIG. 7 depicts the conversion efficiency of aged aqueous derivatized silica media according to one aspect

FIG. 8 depicts the conversion efficiency of freshly produced aqueous derivatized silica media according to one aspect

FIG. 9 depicts the conversion efficiency of 75% solid antioxidant by mass media with antioxidant-derived silica gel according to one aspect

FIG. 10 depicts the conversion efficiency of 50% solid antioxidant by mass media with antioxidant-derived silica gel according to one aspect

FIG. 11 depicts the conversion efficiency of 37% solid antioxidant by mass media with antioxidant-derived silica gel according to one aspect

FIG. 12 depicts the conversion efficiency of 25% solid antioxidant by mass media with antioxidant-derived silica gel according to one aspect

FIG. 13 depicts the conversion efficiency of a 50% solid antioxidant with silica gel without water, according to one aspect.

FIG. 14 depicts the conversion efficiency of 100% solid antioxidants according to one aspect.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

As used herein, the terms “optional” or “optionally” mean that the subsequently described event or circumstance can or cannot occur and that the description includes instances where said event or circumstance occurs and instances where it does not.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used in the description and the appended claims, the singular forms “a,” “an,” and “the” include plural referents unless the context clearly dictates otherwise. Thus, for example, a reference to “a single-use unit” includes not only one but also two or more such units, and a reference to “an apparatus” includes not only one but also two or more such apparatuses and the like.

Throughout the description and claims of this specification, the word “comprise” and other forms of the word, such as “comprising” and “comprises,” are open, non-limiting terms and mean “including but not limited to,” and are not intended to exclude, for example, other additives, segments, integers, or steps. Furthermore, it is to be understood that the terms “comprise,” “comprising,” and “comprises” as they relate to various aspects, elements, and features of the disclosed invention also include the more limited aspects of “consisting essentially of” and “consisting of.”

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate, effective amount will be readily determined by one of ordinary skill in the art.

Unless defined otherwise, all technical and scientific terms used herein have the same meaning as commonly understood by one of ordinary skill in the art to which this invention belongs. In this specification and in the claims which follow, reference will be made to a number of terms that shall be defined herein.

For the terms “for example” and “such as” and grammatical equivalences thereof, the phrase “and without limitation” is understood to follow unless explicitly stated otherwise. It is further understood that these phrases are used for explanatory purposes only. It is further understood that the term “exemplary,” as used herein, means “an example of” and is not intended to convey an indication of a preferred or ideal aspect.

The expressions “ambient temperature” and “room temperature” as used herein are understood in the art and refer generally to a temperature from 20° C. to 35° C.

All disclosed values also include values that fall within +10% variation from the disclosed value unless otherwise indicated or inferred. In other words, if a range of 1 to 10 is disclosed, then a range of about 1 to about 10 is disclosed. In such aspects, it is understood that the amount or value in question can be the exact value or a value that provides equivalent results or effects as recited in the claims or taught herein. That is, amounts, sizes, formulations, parameters, and other quantities and characteristics include both exact values but also approximate, larger or smaller values as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘x, y, z, or less’ and should be interpreted to include the specific ranges of ‘x,’ ‘y,’ ‘z,’ ‘about x,’ ‘about y,’ and ‘about z’ as well as the ranges of ‘less than x,’ ‘less than y, or ‘less than z,’ or ‘less than about x,’ ‘less than about y, and ‘less than about z.’ Likewise, the phrase′ x, y, z, or greater′ should be interpreted to include the specific ranges of ‘x,’ ‘y,’ ‘z,’ ‘about x,’ ‘about y,’ and ‘about z’ as well as the ranges of ‘greater than x,’ greater than y,′ ‘greater than z,’ or ‘greater than about x,’ greater than about y,′ ‘greater than about z.’ In addition, the phrase” ‘x’ to ‘y’,” where ‘x’ and y′ are numerical values, also includes “about x′ to about y′.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5% but also include individual values (e.g., 1%, 2%, 3%, and 4%) and the sub-ranges (e.g., 0.5% to 1.1%; 5% to 2.4%; 0.5% to 3.2%, and 0.5% to 4.4%, and other possible sub-ranges) within the indicated range.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

In still further aspects, when the specific values are disclosed between two end values, it is understood that these end values can also be included.

In still further aspects, when the range is given, and exemplary values are provided, it is understood that any ranges can be formed between any exemplary values within the broadest range. For example, if individual numbers 1, 2, 3, 4, 5, 6, 7, etc. are disclosed, then the ranges 1-7, 2-7, 3-7, 4-7, 5-7, 6-7, 1-6, 1-5, 1-4, 1-3, 1-2, 2-6, 2-5, etc. are also disclosed.

References in the specification and concluding claims to parts by weight of a particular element or component in a composition denote the weight relationship between the element or component and any other elements or components in the composition or article for which a part by weight is expressed. Thus, in a mixture containing 2 parts by weight of component X and 5 parts by weight, components Y, X, and Y are present at a weight ratio of 2:5 and are present in such a ratio regardless of whether additional components are contained in the mixture.

A weight percent (wt. %) of a component, unless specifically stated to the contrary, is based on the total weight of the formulation or composition in which the component is included.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

As used herein, the term “and/or” includes any and all combinations of one or more of the associated listed items.

It will be understood that although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example aspects.

As used herein, the term “substantially” means that the subsequently described event or circumstance completely occurs or that the subsequently described event or circumstance generally, typically, or approximately occurs.

Still further, the term “substantially” can, in some aspects, refer to at least 90%, at least 95%, at least 99%, or 100% of the stated property, component, composition, or other condition for which substantially is used to characterize or otherwise quantify an amount.

In other aspects, as used herein, the term “substantially free,” when used in the context of a composition or component of a composition that is substantially absent, is intended to refer to an amount that is then 1% by weight, e.g., less than 0.5% by weight, less than 0.1% by weight, less than 0.05% by weight, or less than 0.01% by weight of the stated material, based on the total weight of the composition.

As used herein, “treating” and “treatment” generally refer to obtaining a desired pharmacological or physiological effect. The effect can be but does not necessarily have to be prophylactic in preventing or partially preventing a disease, symptom, or condition. The effect can be therapeutic regarding a partial or complete cure of a disease, condition, symptom, or adverse effect attributed to the disease, disorder, or condition. The term “treatment” as used herein can include any treatment of a disorder in a subject, particularly a human. It can include any one or more of the following: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease or its symptoms or conditions. The term “treatment,” as used herein, can refer to both therapeutic treatment alone, prophylactic treatment alone, or both therapeutic and preventive treatment. Those in need of treatment (i.e., subjects in need thereof) can include those already with the disorder or those in which the disorder is to be prevented. As used herein, the term “treating” can include inhibiting the disease, disorder, or condition, e.g., impeding its progress; and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

Some aspects described herein relate to methods. It should be understood that such methods can be computer-implemented. That is, where the method or other events are described herein, it should be understood that they may be performed by a computing device having a processor and a memory. Memory of a computing device is also referred to as a non-transitory computer-readable medium, which can include instructions or computer code for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also referred to as code) may be those designed and constructed for a specific purpose or purpose. Examples of non-transitory computer-readable media include but are not limited to magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules, Read-Only Memory (ROM), Random-Access Memory (RAM) and/or the like. One or more processors can be communicatively coupled to the memory and operable to execute the code stored on the non-transitory processor-readable medium. Examples of processors include general purpose processors (e.g., CPUs), Graphical Processing Units, Field Programmable Gate Arrays (FPGAs), Application Specific Integrated Circuits (ASICs), Digital Signal Processor (DSPs), Programmable Logic Devices (PLDs), and the like. Examples of computer code include, but are not limited to, micro-code or micro-instructions, machine instructions, such as those produced by a compiler, code used to produce a web service, and files containing higher-level instructions that are executed by a computer using an interpreter. For example, aspects may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include but are not limited to, control signals, encrypted code, and compressed code.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

The present invention may be understood more readily by reference to the following detailed description of various aspects of the invention and the examples included therein.

### Device

Various systems and devices for generating nitric oxide (NO) are disclosed herein. Generally, NO is inhaled or otherwise delivered to a patient's lungs. Since NO is inhaled, much higher local doses can be achieved without concomitant vasodilation of the other blood vessels in the body. Accordingly, NO gas having a concentration of 0.1 ppm to 1000 ppm (e.g., 0.1, 0.5, 1, 5, 10, 40, 80, 100, 150, 200, 250, 300, 350, 400, 450, 500, 550, 600, 650, 700, 750, 800, 850, 900, 950, or 1000 ppm) may be delivered to a patient. In still further aspects, the NO gas can have a concentration that falls between any two foregoing values or within a range formed by any two foregoing values. For example, NO gas can have a concentration of 0.1 ppm to 1000 ppm, 0.1 ppm to 900 ppm, 0.1 ppm to 800 ppm, 0.1 ppm to 700 ppm, 0.1 ppm to 600 ppm, 0.1 ppm to 500 ppm, 0.1 ppm to 400 ppm, 0.1 ppm to 300 ppm, 0.1 ppm to 200 ppm, 0.1 ppm to 100 ppm, 0.1 ppm to 80 ppm, 0.1 ppm to 50 ppm, 0.1 ppm to 20 ppm, 0.1 ppm to 10 ppm, 0.1 ppm to 5 ppm, 0.1 ppm to 1 ppm, 0.1 ppm to 0.5 ppm, 0.5 ppm to 1000 ppm, 1 ppm to 1000 ppm, 5 ppm to 1000 ppm, 10 ppm to 1000 ppm, 20 ppm to 1000 ppm, 50 ppm to 1000 ppm, 80 ppm to 1000 ppm, 100 ppm to 1000 ppm, 200 ppm to 1000 ppm, 300 ppm to 1000 ppm, 400 ppm to 1000 ppm, 500 ppm to 1000 ppm, 60 ppm to 1000 ppm, 700 ppm to 1000 ppm, or 800 ppm to 1000 ppm. Yet in other aspects, the NO concentration can be greater than 1000 ppm, greater than 1200 ppm, greater than 1500 ppm, greater than 1750 ppm, or even equal to or greater than 2000 ppm.

Accordingly, high doses of NO may be used to prevent, reverse, or limit the progression of disorders, which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma, status asthmaticus, or hypoxia. NO can also be used to treat chronic pulmonary hypertension, bronchopulmonary dysplasia, chronic pulmonary thromboembolism, idiopathic pulmonary hypertension, primary pulmonary hypertension, or chronic hypoxia.

Currently, approved devices and methods for delivering inhaled NO gas require complex and heavy equipment. NO gas is stored in heavy gas bottles with nitrogen and no traces of oxygen. NO gas is mixed with air or oxygen with specialized injectors and complex ventilators, and the mixing process is monitored with equipment having sensitive microprocessors and electronics. All this equipment is required in order to ensure that NO is not oxidized into nitrogen dioxide (NO₂) during the mixing process since NO₂ is highly toxic. However, this equipment is not conducive to use in a non-medical facility setting since the size, cost, complexity, and safety issues restrict the operation of this equipment to highly-trained professionals in a medical facility.

Some of the devices for forming nitric oxide from NO₂ are disclosed in U.S. Pat. Nos. 8,607,785, 8,944,049, 9,604,028, 10,926,054, 11,744,978, the contents of which are incorporated herein in their whole entirety.

In contrast, the devices and systems disclosed herein do not require the storage of nitric oxide in heavy gas bottles. The devices disclosed herein allow the formation of nitric oxide from nitrogen dioxide on demand, when a predetermined amount of nitric oxide is needed to be delivered to a patient.

In certain aspects, disclosed herein is a device comprising a media configured to convert nitrogen dioxide into nitric oxide, wherein the media comprises: (a) a support material; and (b) an antioxidant material, wherein at least 15 wt % of the antioxidant is material present in a solid form. In such exemplary and unlimiting aspects, the wt % is calculated based on a total weight of the antioxidant material present in the system. In certain aspects, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, or 100 wt %, of the antioxidant material is present in a solid form. In still further aspects, an amount of the antioxidant material present in the solid form is 15 wt % to 100 wt %, 20 wt % to 100 wt %, 25 wt % to 100 wt %, 35 wt % to 100 wt %, 45 wt % to 100 wt %, 55 wt % to 100 wt %, 65 wt % to 100 wt %, 75 wt % to 100 wt %, 85 wt % to 100 wt %, 95 wt % to 100 wt %, 15 wt % to 95 wt %, 15 wt % to 85 wt %, 15 wt % to 75 wt %, 15 wt % to 65 wt %, 15 wt % to 55 wt %, 15 wt % to 45 wt %, 15 wt % to 35 wt %, or 15 wt % to 25 wt % and so on.

In certain aspects, the antioxidant material is present only in the solid form.

In certain aspects, the antioxidant material is present in an amount greater than 0 wt % to less than 100 wt % based on the total weight of the media. In yet still further aspects, the antioxidant material is present in an amount greater than 0 wt % to less than 100%, greater than 0 wt % to 90 wt %, greater than 0 wt % to 80 wt %, greater than 0 wt % to 70 wt %, greater than 0 wt % to 60 wt %, greater than 0 wt % to 50 wt %, greater than 0 wt % to 40 wt %, greater than 0 wt % to 30 wt %, greater than 0 wt % to 20 wt %, greater than 0 wt % to 10 wt %, 1 wt % to less than 100%, 5 wt % to less than 100%, 10 wt % to less than 100%, 20 wt % to less than 100%, 25 wt % to less than 100%, 30 wt % to less than 100%, 40 wt % to less than 100%, 50 wt % to less than 100%, 60 wt % to less than 100%, 70 wt % to less than 100%, 80 wt % to less than 100%, and so on based on the total weight of the media.

Yet in still further aspects, the media can further comprise a solvent present in an amount of 0 wt % to 40 wt % based on the total weight of the media, including exemplary values of 0.01 wt %, 0.05 wt %, 0.1 wt %, 0.5 wt %, 1 wt %, 5 wt %, 10 wt %, 15 wt %, 20 wt %, 25 wt %, 30 wt %, and 35 wt %. It is understood that solvent can be present in any amount that falls within any two foregoing values or within a range formed by any two foregoing values. For example, the solvent can be present in an amount 0 wt % to 40 wt %, 0.05 wt % to 40 wt %, 0.5 wt % to 40 wt %, 1 wt % to 40 wt %, 5 wt % to 40 wt %, 10 wt % to 40 wt %, 20 wt % to 40 wt %, 0 wt % to 35 wt %, 0 wt % to 30 wt %, 0 wt % to 25 wt %, 0 wt % to 20 wt %, 0 wt % to 10 wt %, 0 wt % to 1 wt %, 0 wt % to 0.5 wt %, and so on based on the total weight of the media.

It is understood that in aspects disclosed herein the solvent can comprise any fluid or liquid that fits the desired application. For example, and without limitations the solvent can comprise water, an organic solvent, or a combination thereof.

In still further aspects, if the solvent is present in any of the disclosed above amounts, the media can comprise a mixture of the antioxidant material present in the solid form and the antioxidant dissolved in the solvent. In yet still further aspects, the antioxidant can be present as a saturated solution of the solvent and the solid antioxidant. In yet still further aspects, and as described above, it is understood that at least 15 wt % of the total amount of the antioxidant material is present in the solid form.

In still further aspects, in still further aspects, the antioxidant can be present in any known solid form. In still further aspects, the antioxidant material present in the solid form has a particle size of 1 to 1000 microns, including exemplary values of 2 microns, 5 microns, 10 microns, 20 microns, 50 microns, 100 microns, 200 microns, 400 microns, 500 microns, 600 microns, and 900 microns. It is understood that the particle size can fall between any two foregoing values or within a range formed by any two foregoing values. For example and without limitations, the antioxidant material present in the solid form has a particle size of 1 to 1000 microns, 1 to 900 microns, 1 to 800 microns, 1 to 700 microns, 1 to 600 microns, 1 to 500 microns, 1 to 400 microns, 1 to 300 microns, 1 to 200 microns, 1 to 100 microns, 1 to 50 microns, 1 to 20 microns, 1 to 10 microns, 1 to 5 microns, 5 to 1000 microns, 10 to 1000 microns, 25 to 1000 microns, 50 to 1000 microns, 75 to 1000 microns, 100 to 1000 microns, 250 to 1000 microns, 300 to 1000 microns, 500 to 1000 microns, 700 to 1000 microns, and so on.

In still further aspects, the antioxidant material can comprise ascorbic acid; alpha-, beta-, gamma-, or delta tocopherol; alpha-, beta-, gamma-, or delta-tocotrienol; polyphenols; beta-carotene; or a combination thereof. It is understood that any other antioxidant materials suitable for the desired application and capable of being present in solid form can be utilized.

Ins till further aspects, the support material can comprise a molecular sieve, a metal-organic framework (MOF), natural zeolites, synthetic zeolites, polymers of intrinsic microporosity (PIMs), hyper-crosslinked microporous polymers (HCPs), covalent organic frameworks (COFs), conjugated microporous polymers (CMPs), porous aromatic frameworks (PAFs), porous organic cages (PCs), silica gel, or any combination thereof.

In certain aspects, the specific surface area of the support material is 350 to 6000 m²/g, including exemplary values of 400 m²/g, 500 m²/g, 600 m²/g, 700 m²/g, 800 m²/g, 900 m²/g, 1000 m²/g, 1250 m²/g, 1500 m²/g, 1750 m²/g, 2000 m²/g, 2250 m²/g, 2500 m²/g, 2750 m²/g, 3000 m²/g, 3250 m²/g, 3500 m²/g, 3750 m²/g, 4000 m²/g, 4250 m²/g, 4500 m²/g, 4750 m²/g, 5000 m²/g, 5250 m²/g, 5500 m²/g, 5750 m²/g, and 5990 m²/g. It is understood that the specific surface area can be in any range formed between any two foregoing values. For example, and without limitations, the specific surface area can be 350 to 4500 m²/g, 400 to 6000 m²/g, 400 to 5000 m²/g, 500 to 6000 m²/g, or 500 to 5000 m²/g, or 500 to 4000 m²/g or 500 to 3000 m²/g, 400 to 3000 m²/g, 400 to 2000 m²/g, 1000 to 6000 m²/g, and so on.

In still further aspects, the support material can be a macroporous, mesoporous, or microporous material. In certain aspects, the support material is macroporous. In other aspects, the support material is mesoporous, in still further aspects, the support material is microporous. In yet still further aspects, the support material can be a mixture of macroporous portions and/or mesoporous portions, and/or microporous portions. In still further aspects, the support material can comprise a continuous polymer phase permeated by a continuous pore phase.

It is understood that the term “continuous” in the context of polymer phase or pore phase generally refers to a phase such that all points within the phase are directly connected so that for any two points within a continuous phase, there exists a path which connects the two points without leaving the phase.

In still further aspects, the support material disclosed herein can have a pore size of 1 angstrom to 1000 angstroms, including exemplary values of 2 angstroms, 5 angstroms, 10 angstroms, 15 angstroms, 20 angstroms, 25 angstroms, 30 angstroms, 35 angstroms, 40 angstroms, 50 angstroms, 60 angstroms, 70 angstroms, 80 angstroms, 90 angstroms, 100 angstroms, 200 angstroms, 300 angstroms, 400 angstroms, 500 angstroms, 600 angstroms, 700 angstroms, 800 angstroms, and 900 angstroms. It is understood that the pore size can be in any range formed between any two foregoing values. For example, and without limitations, the pore size can be 1 to 1000 angstroms, 1 to 500 angstroms, or 1 to 200 angstroms, or 1 to 100 angstroms, or 1 to 50 angstroms, or 1 to 40 angstroms, or 1 to 30 angstroms, and so on.

In still further aspects, the support material has a pore size of 1 angstrom to 100 angstrom, including exemplary values of 2 angstroms, 5 angstroms, 10 angstroms, 15 angstroms, 20 angstroms, 25 angstroms, 30 angstroms, 35 angstroms, 40 angstroms, 50 angstroms, 60 angstroms, 70 angstroms, 80 angstroms, and 90 angstroms. It is understood that the pore size can be in any range formed between any two foregoing values. For example, and without limitations, the pore size can be 1 to 100 angstroms, or 1 to 80 angstroms, or 1 to 70 angstroms, or 1 to 50 angstroms, or 1 to 40 angstroms, or 1 to 30 angstroms, and so on.

In still further aspects, the support material has a pore size of 1 angstrom to 50 angstrom, including exemplary values of 2 angstroms, 3 angstroms, 4 angstroms, 5 angstroms, 6 angstroms, 7 angstroms, 8 angstroms, 9 angstroms, 10 angstroms, 11 angstroms, 12 angstroms, 13 angstroms, 14 angstroms, 15 angstroms, 16 angstroms, 17 angstroms, 18 angstroms, 19 angstroms, 20 angstroms, 21 angstroms, 22 angstroms, 23 angstroms, 24 angstroms, 25 angstroms, 26 angstroms, 27 angstroms, 28 angstroms, 29 angstroms, 30 angstroms, 31 angstroms, 32 angstroms, 33 angstroms, 34 angstroms, 35 angstroms, 36 angstroms, 37 angstroms, 38 angstroms, 39 angstroms, 40 angstroms, 41 angstroms, 42 angstroms, 43 angstroms, 44 angstroms, 45 angstroms, 46 angstroms, 47 angstroms, 48 angstroms, and 49 angstroms. In still further aspects, a pore size of less than 40 angstroms. It is understood that the pore size can be in any range formed between any two foregoing values. For example, and without limitations, the pore size can be 1 to 50 angstroms, or 1 to 45 angstroms, or 1 to 40 angstroms, or 1 to 35 angstroms, or 1 to 30 angstroms, or 1 to 25 angstroms, or 1 to 20 angstroms, or 1 to 15 angstroms, or 1 to 10 angstroms, and so on.

In certain aspects, the support material can comprise natural and/or synthetic zeolites. In yet other aspects, the support material can comprise a molecular sieve. In yet other aspects, the support material can comprise silica gel. In still further aspects, the support material can comprise MOFs. For example, and without limitations, MOFs can be Al-based, Ti-based, Zr-based, Ni-based, Co-based, and so on, and any combination thereof.

In still further aspects, the support material can comprise polymers of intrinsic microporosity (PIMs). It is understood that any known PIMs can be utilized. For example, and without limitations, any PIMs disclosed in “Polymer of Intrinsic Microporosity” by N. B. Mckeown (International Scholarly Research Network, volume 2012, article ID 513986, 16 pages, doi: 10.5402/2012/513986), U.S. Pat. No. 8,623,928, WO2003000774 and/or WO2005012397, each of which is hereby incorporated by reference herein in its entirety for its teachings on polymers of intrinsic microporosity.

In still further aspects, the support material can comprise hyper-crosslinked microporous polymers (HCPs). Any known in the art HCPs can be used. For example, and without limitations, hyper-crosslinked microporous polymers include Tetraphenyl anthraquinone-based HCP, Binaphthol-based HCP, and A porous organic polymer containing triazine and carbazole moieties. Some additional and unlimiting examples can be found in Polymers, 2017, 9(12), 651 by R. Castaldo et al. in “Microporous Hyper-Crosslinked polystyrenes and nanocomposites with high adsorption properties: A Review,” which is hereby incorporated by reference herein in its entirety.

In still further aspects, the support material can comprise covalent organic frameworks (COFs). Any known in the art COFs can be used. Some unlimiting examples of such materials and methods of making the same are described in Giant, 6, 2021, by H. R. Abuzeid et al. in “Covalent organic frameworks: Design principles, synthetic strategies, and diverse applications,” or Polymers, 2021, 13(6), 970, by T. F. Machado et al. in “Covalent Organic Frameworks: Synthesis, Properties and Applications—An Overview,” each of which is hereby incorporated by reference herein in its entirety for its teachings on covalent organic frameworks.

In still further aspects, the support material can comprise conjugated microporous polymers (CMPs). Any known in the art CMPs can be used. Some unlimited examples can be found in Chemical Reviews, 2020, 120, 2171-2214 by J-Sing M. Lee et al. in “Advances in Conjugated Microporous Polymers,” which is hereby incorporated by reference herein in its entirety.

In still further aspects, the support material can comprise porous aromatic frameworks (PAFs). Any known in the art PAFs can be used. Some unlimited examples can be found in Chemical Reviews, 2020, 120, 16, 8934-8986 by Y. Tian et al. in “Porous Aromatic Frameworks,” which is hereby incorporated by reference herein in its entirety.

In still further aspects, the support material can comprise porous organic cages (PCs). Any known in the art PCs can be used. Some unlimited examples can be found in Chemical Reviews, 2023, 123, 8, 4602-4634 by X. Yang et al. in “Porous Organic Cages,” which is hereby incorporated by reference herein in its entirety.

In still further aspects, the antioxidant material is embedded (or impregnated) within at least a portion of the support material. Yet in still further aspects, the antioxidant material is substantially uniformly embedded within the support material. Yet in still further aspects, the antioxidant material is homogeneously mixed with the support material and distributed on the surface and within the pores on the support material.

It is understood that the support materials disclosed herein allow for a more efficient interaction between the antioxidant and N₂O₄ and/or NO₂ to produce nitric oxide.

An exemplary reaction between the nitrogen dioxide and the media disclosed herein is shown below.

$$\begin{matrix}a . & 6 {NO}_{2} ( gas ) + 3 {H}_{2} O ( liq . ) \to 3 {HNO}_{3} ( liq . ) + 3 {HNO}_{2}\end{matrix} ( liq . ) \qquad Eq . 1 a$$

$$\begin{matrix}b . & 3 {HNO}_{2} ( liq . ) \to {HNO}_{3} ( liq . ) + 2 NO ( gas ) + H 2 O\end{matrix} ( liq . ) \qquad Eq . 1 b$$

Yet in other aspects, the NO can be formed according to Eq. 2a-2b:

$${C}_{6} {H}_{8} {O}_{6} + {NO}_{2} = > {C}_{6} {H}_{6} {O}_{6} + NO + {H}_{2} O \qquad Eq . 2 a$$

$$3 {NO}_{2} + {H}_{2} O = > 2 {HNO}_{3} + NO \qquad Eq . 2 b$$

In still further aspects, the antioxidant material is present in an effective amount to form a predetermined amount of nitric oxide. In yet still, in further aspects, the support material is present in an effective amount to form a predetermined amount of nitric oxide.

In still further aspects, the media disclosed herein can comprise a polymeric material. In such aspects, the polymeric material can be different from the polymeric material present in the support material. In still further aspects, the polymeric material can be a thermoplastic material. In yet still further aspects, the polymeric material can comprise polyethylene, polypropylene, polyamide, polyurethane, polystyrene, or any combination thereof.

Any of the disclosed above antioxidants and/or support materials can be used to form the nitric oxide in the desired amount.

In still further aspects, the media exhibits conversion efficiency greater than 20%, greater than 30%, greater than 40%, greater than 50%, greater than 60%, greater than 70%, greater than 80%, or greater than 90%. In yet other aspects, the media exhibits conversion efficiency of at least 20%, at least 30%, at least 40%, at least 50%, at least 60%, at least 70%, at least 80%, at least 90%, or at least 95%. In yet other aspects, the media exhibits conversion efficiency is 20% to 100%, 20% to 90%, 20% to 80%, 20% to 70%, 30% to 100%, 40% to 100%, 50% to 100%, 60% to 100%, 70% to 100%, 80% to 100%, 90% to 100%, 60% to 90%, 60% to 95%, and so on. It is understood that the term conversion efficiency refers to an efficiency of converting NO₂ to NO. It is also understood that NO₂ source can be present as liquid N₂O₄.

In still further aspects, the device described herein is configured to achieve any desired dosage by controlling the dosing rate. In still further aspects, the device is capable of dosing up to 2000 ppm-LPM (liters per minute), up to 2200 ppm-LPM, up to 2400 ppm-LPM, up to 2600 ppm-LPM, up to 2800 ppm-LPM, up to 3000 ppm-LPM, up to 3200 ppm-LPM, up to 3400 ppm-LPM, up to 3600 ppm-LPM, up to 3800 ppm-LPM, up to 4000 ppm-LPM, up to 4200 ppm-LPM, up to 4400 ppm-LPM, up to 4600 ppm-LPM, up to 4800 ppm-LPM, up to 5000 ppm-LPM, up to 5200 ppm-LPM, up to 5400 ppm-LPM, up to 5600 ppm-LPM, up to 5800 ppm-LPM, up to 6000 ppm-LPM, up to 6200 ppm-LPM, up to 6400 ppm-LPM, up to 6600 ppm-LPM, up to 6800 ppm-LPM, or up to 7000 ppm-LPM. In still further aspects, the device is capable of dosing up to 6400 ppm-LPM.

In still further aspects, the media is disposed within a vessel. In some exemplary and unlimiting aspects, the media is formed within the vessel. In yet other aspects, the media can conform to the vessel's dimensions and shape. In still further aspects, the media is formed into a shape conforming with the vessel. In still further aspects, the media is present in a granular form that fills the vessel's dimensions in an effective amount relative to the volume of the vessel.

In yet still further aspects, the media is present in a gel form that fills the vessel's dimensions in an effective amount relative to the volume of the vessel.

In still further aspects, the media is formed into a shape conforming with the vessel.

In still further aspects, the media can also comprise a gel. In certain aspects, the gel can be a hydrogel. In still further aspects, the gel can comprise the support material and the antioxidant dispersed within it. In still further aspects, the media can comprise a suspension. In still further aspects, the media is present in a solid form, a granular form, a gel, a liquid form, or any combination thereof.

In certain exemplary and unlimiting aspects, the vessel containing the disclosed herein media is a pressure vessel. It is understood that the term “pressure vessel” as used herein refers to any vessel capable of containing (a liquid and/or gas and/or fluid and/or gel or solid) and/or withstanding pressure up to 1000 psi, up to 900 psi, up to 800 psi, up to 700 psi, up to 600 psi, or up to 500 psi. In still further aspects, the pressure vessel as described herein is capable of containing (a liquid and/or gas and/or fluid) and/or withstanding pressure of equal to or greater than 14 psi to 1000 psi, including exemplary values of 15 psi, 50 psi, 100 psi, 150 psi, 200 psi, 250 psi, 300 psi, 350 psi, 400 psi, 450 psi, 500 psi, 550 psi, 600 psi, 650 psi, 700 psi, 750 psi, 800 psi, 850 psi, 900 psi, and 950 psi. It is understood that any ranges between any two foregoing values can be formed. For example, and without limitations, the pressure vessel as described herein is capable of containing a liquid and/or gas and/or fluid and/or withstanding pressure of 15 psi to 600 psi, 15 psi to 500 psi, or 15 psi to 400 psi, or 100 psi to 900 psi, and so on. It is understood that the first chamber is made of a material that is capable of withstanding the disclosed pressures and chemical conditions in which the chamber operates. In certain exemplary and limiting aspects, the first vessel can be made of stainless steel, aluminum, inductive metals, alloys thereof, thermally conductive polymers, or any combination thereof.

In still further aspects, the media is in fluid communication with a source of NO₂. It is understood that the source of NO₂ can be any source. For example, in some aspects, the source of NO₂ can be liquid N₂O₄. In other aspects, the source of NO₂ can be N₂O₄ incorporated in a matrix, for example gel. In other aspects, it is understood that N₂O₄ can be in equilibrium with NO₂. In still further aspects, the source of NO₂ can be a gas tank comprising NO₂, and so on.

In still further aspects, the media comprises a fluidic pathway configured to transfer formed nitric oxide to a patient.

Also disclosed herein is a system for forming nitric oxide comprising: a source of NO₂; a vessel comprising any of the disclosed herein media a patient interface coupled to the vessel and configured to deliver the nitric oxide to a patient. In still further aspects, the system comprises one or more humidifiers that are in communication with the vessel. In still further aspects, the system comprises a control unit and one or more sensors configured to measure an amount of formed nitric oxide and/or an amount of nitrogen dioxide. The system can further comprise inerting chambers configured to inert residual or leaked nitrogen dioxide and/or N₂O₄.

### Methods

Also disclosed herein are methods of making the disclosed herein media, devices and systems.

In certain aspects, disclosed are methods where all components are dry blended. For example, and without limitations, the methods comprise dry blending of any of the disclosed above antioxidants present in the solid form with any of the disclosed above support materials.

In certain aspects, the support material can comprise a hydrated material. In other words, the support material can comprise some effective amount of water or water vapor.

Yet in other aspects, also disclosed are methods comprising mixing a saturated or supersaturated solution of any of the disclosed above antioxidants in any of the disclosed above solvents with any of the disclosed above support materials. Such methods can further comprise, removing the solvent to form the antioxidant material present in the solid form.

### EXAMPLES

The following examples are set forth below to illustrate the methods and results according to the disclosed subject matter. These examples are not intended to be inclusive of all aspects of the subject matter disclosed herein but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, the temperature is in ° C. or is at ambient temperature, and pressure is at or near atmospheric. There are numerous variations and combinations of reaction conditions, e.g., component concentrations, temperatures, pressures, and other reaction ranges and conditions, which can be used to optimize the product purity and yield obtained from the described process. Only reasonable and routine experimentation will be required to optimize such process conditions.

### Example 1

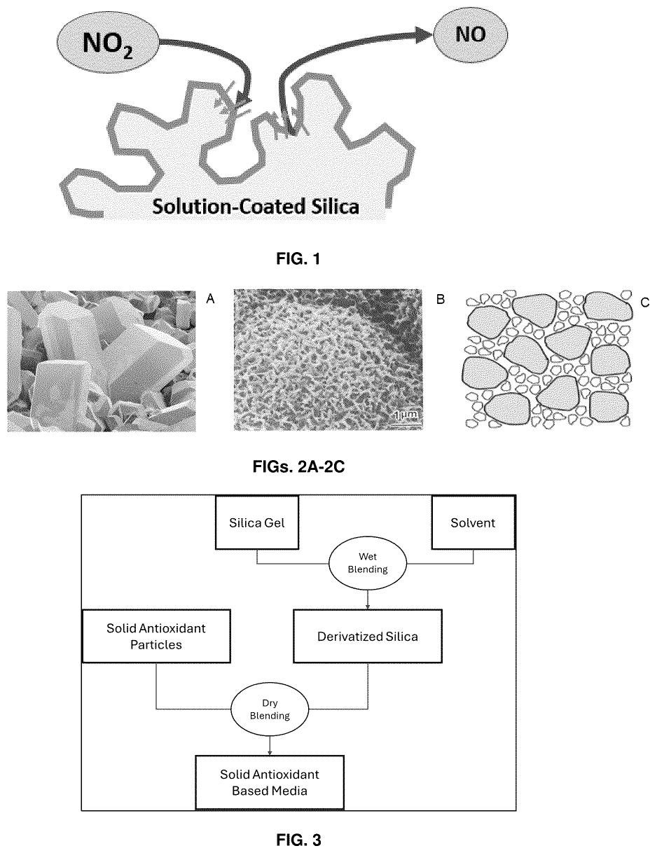

FIG. 1A shows a schematic of forming NO gas in the media where the support material is coated with a solution-based antioxidant. It is understood and without wishing to be bound by any theory that, in such an example, the antioxidant material, if present in an aqueous solution, can have certain challenges. For example, the concentration of antioxidant material can be limited by its solubility in water. The dissolution in water can also affect the rate of degradation of antioxidant materials (to accelerate it when compared to a solid-state). Side reactions, such as the formation of nitric acid, can also occur.

These possible challenges have motivated the inventors to look for alternative solutions that are described herein.

In certain aspects, disclosed herein are devices where the media comprises support material (FIG. 2B), such as for example silica and a solid antioxidant material (FIG. 2A) that can be blended together (FIG. 2C) such that antioxidant material remains in the solid state. Without wishing to be bound by any theory, it is hypothesized that such media will have enhanced stability, minimal side reactions, maximized utilization of the reactants, and improved packing to reduce dose output variability.

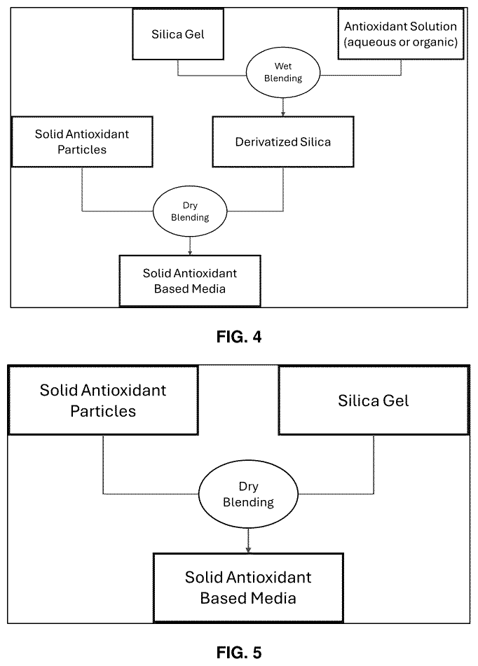

FIGS. 3-6 show various exemplary and unlimiting methods for producing the solid antioxidant-based cartridge media. For example, as shown in FIG. 3, the support material (for example, silica gel) is mixed with any of the disclosed above solvents to form a derivatized silica. Such silica is then mixed by dry blending with a solid antioxidant material particle to form the solid antioxidant-based media.

FIG. 4 shows an alternative exemplary method. In these methods, the silica gel (the support material) is wet blended with an antioxidant solution (aqueous or organic) to form a derivatized silica.

FIG. 5 shows an alternative exemplary method. In this method the support material (for example, silica gel) is dry blended with the solid antioxidant particles.

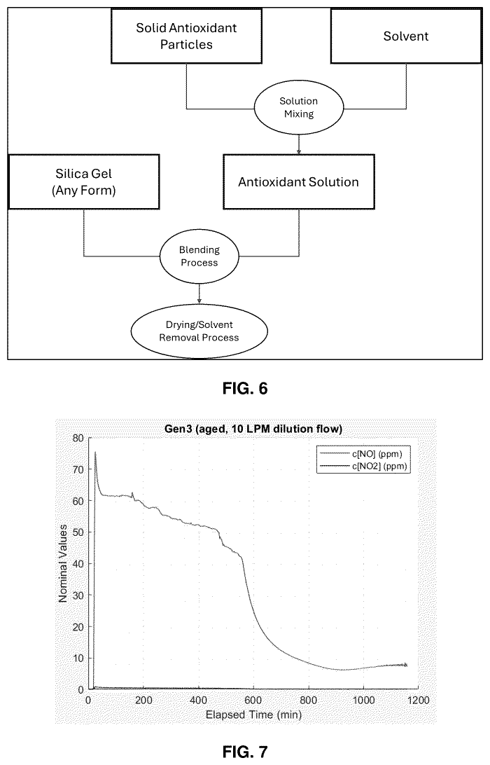

FIG. 6 shows an additional exemplary method. Here, the solids antioxidant particles are mixed with any of the disclosed above solvents to form a solution. This antioxidant solution is then mixed with the support material present in any form, and then the solvent is removed to ensure that at least 15 wt % of the antioxidant material is present in solid form.

By incorporating solid antioxidants into the cartridge, the dominating drug-producing reaction shifts from the aqueous phase to the solid phase. In such a reaction scheme, NO₂ becomes ionized from contact with traces of water, either as vapor or on the surface of the silica gel, which is converted to NO upon contact with solid antioxidants.

The use of solid antioxidants offers several advantages to the derivatized silica-based media, including improved material packing from increased particle size distribution and improved shelf life. The addition of smaller antioxidant particles into the silica gel media improves the packing efficiency, which, in turn, improves the flow path through the cartridge and reduces variations in dosing. Further, the separation of the antioxidant from the water by phase reduces the degradation of the antioxidant, improving the cartridge media shelf life.

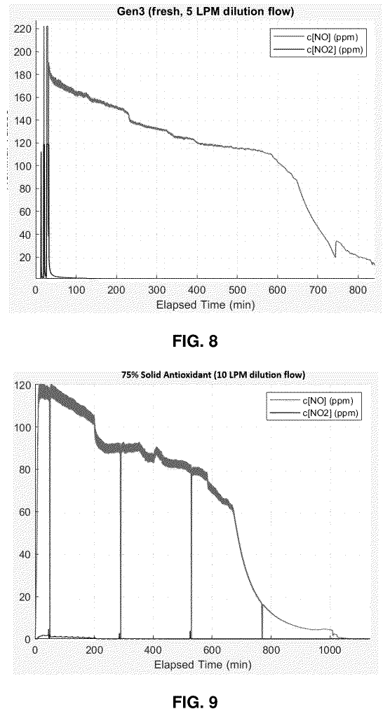

FIGS. 7-8 show NO conversion with 100% aqueous antioxidant media. In certain aspects, the antioxidant is dissolved in water prior to formulating the final mixture. In such aspects, the antioxidant is dissolved in water and then sprayed onto the silica gel (support material).

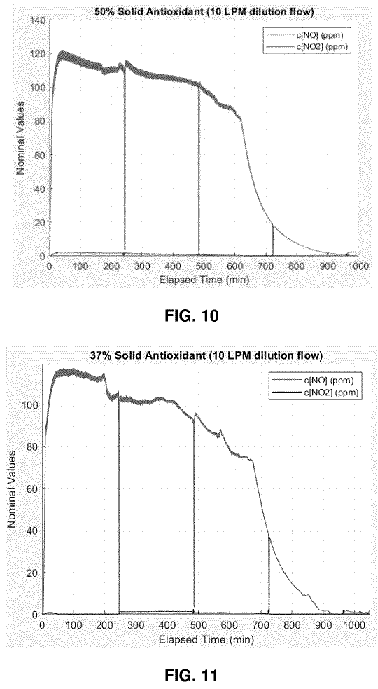

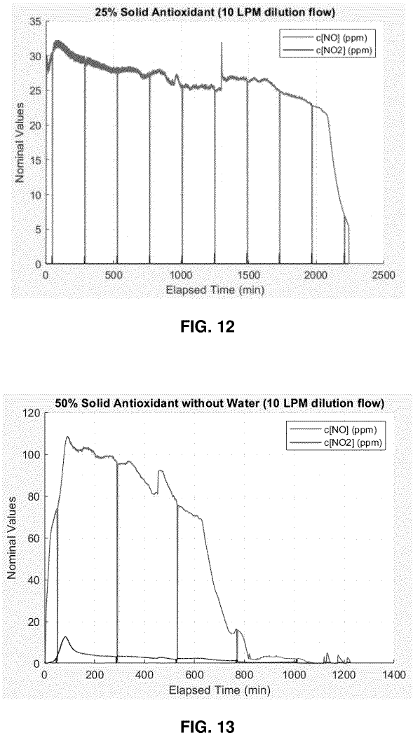

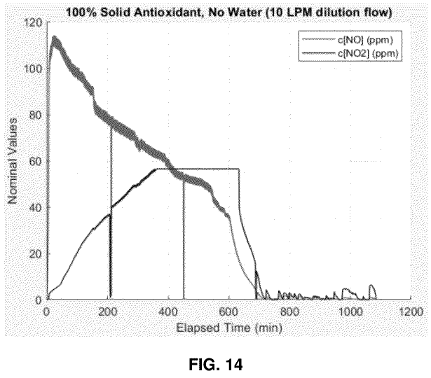

FIGS. 9-14 show high conversion of the disclosed herein antioxidants with an antioxidant content by mass of 25% up to 100% solid antioxidant. Water content ranging from 0% to 30% has shown potential for high conversion efficiency.

It was found that moving to the solid antioxidant materials present in the media can increase ppm-LPM output to 4363 ppm-LPM. It is further found that the conversion efficiency in the system can be as low as 80% and higher than 95% compared to 40-60% when 100% aqueous antioxidant solution is utilized.

While various aspects have been described above, it should be understood that they have been presented by way of example only and not limitation. Furthermore, although various aspects have been described as having particular features and/or combinations of components, other aspects possibly have a combination of any features and/or components from any of the aspects where appropriate, as well as additional features and/or components.

Where methods described above indicate certain events occurring in a certain order, the ordering of certain events may be modified. Additionally, certain of the events may be performed concurrently in a parallel process, when possible, as well as performed sequentially as described above. Although various aspects have been described as having particular features and/or combinations of components, other aspects possibly have a combination of any features and/or components from any of the aspects where appropriate.

### EXEMPLARY ASPECTS

Example 1. A device comprising: a media configured to convert nitrogen dioxide into nitric oxide, wherein the media comprises: (a) a support material; and (b) an antioxidant material, wherein at least 15 wt % of the antioxidant material is present in a solid form.

Example 2. The device of any of the examples herein, particularly Example 1, wherein the media further comprises a solvent present in an amount of 0 wt % to 40 wt % based on the total weight of the media.

Example 3. The device of any of the examples herein, particularly Example 2, wherein the solvent comprises water, an organic solvent, or a combination thereof.

Example 4. The device of any of the examples herein, particularly Examples 1-3, wherein the antioxidant material is present in an amount greater than 0 wt % to less than 100% based on the total weight of the media.

Example 5. The device of any of the examples herein, particularly Examples 1-4, wherein the antioxidant material present in the solid form is present in an amount greater than 50 wt %.

Example 6. The device of any of the examples herein, particularly Examples 1-4, wherein the antioxidant material is present only in the solid form.

Example 7. The device of any of the examples herein, particularly Examples 2-5, wherein when the solvent is present in an amount greater than 0 wt % to 40 wt %, the media further comprises a mixture of the antioxidant material present in the solid form and the antioxidant dissolved in the solvent.

Example 8. The device of any of the examples herein, particularly Examples 1-7, wherein the antioxidant material present in the solid form has a particle size of 1 to 1000 microns.

Example 9. The device of any of the examples herein, particularly Examples 1-8, wherein the antioxidant material comprises ascorbic acid; alpha-, beta-, gamma-, or delta tocopherol; alpha-, beta-, gamma-, or delta-tocotrienol; polyphenols; beta-carotene; or a combination thereof.

Example 10. The device of any of the examples herein, particularly Examples 1-9, wherein the support material comprises a molecular sieve, a metal-organic framework (MOF), natural zeolites, synthetic zeolites, polymers of intrinsic microporosity (PIMs), hyper-crosslinked microporous polymers (HCPs), covalent organic frameworks (COFs), conjugated microporous polymers (CMPs), porous aromatic frameworks (PAFs), porous organic cages (PCs), silica gel, or any combination thereof.

Example 11. The device of any of the examples herein, particularly Examples 1-10, wherein the support material has a particle size of 1 to 1000 micrometers.

Example 12. The device of any of the examples herein, particularly Examples 1-11, wherein the support material is a porous material, has an average pore size of 1 to 1000 angstroms.

Example 13. The device of any of the examples herein, particularly Examples 1-12, wherein the support material exhibits a specific surface area of 400 to 6000 m²/g.

Example 14. The device of any of the examples herein, particularly Examples 1-13, wherein the media exhibits conversion efficiency greater than 60%.

Example 15. The device of any of the examples herein, particularly Examples 1-14, wherein the media exhibits conversion efficiency greater than 80%.

Example 16. The device of any of the examples herein, particularly Examples 1-15, wherein the media exhibits conversion efficiency greater than 90%.

Example 17. The device of any of the examples herein, particularly Examples 1-16, wherein the antioxidant material is present in an effective amount to form a predetermined amount of nitric oxide.

Example 18. The device of any of the examples herein, particularly Examples 1-17, wherein the support material is present in an effective amount to form a predetermined amount of nitric oxide.

Example 19. The device of any of the examples herein, particularly Examples 1-16, wherein the device is capable of dosing up to 6400 ppm-LPM.

Example 20. The device of any of the examples herein, particularly Examples 1-19, wherein the media is disposed within a vessel.

Example 21. The device of any of the examples herein, particularly Example 20, wherein the media is compounded within the vessel.

Example 22. The device of any of the examples herein, particularly Example 21, wherein the media conforms to the vessel's dimensions.

Example 23. The device of any of the examples herein, particularly Examples 1-22, wherein the media is in fluid communication with a source of NO₂.

Example 24. The device of any of the examples herein, particularly Examples 1-23, wherein the media comprises a fluidic pathway configured to transfer formed nitric oxide to a patient.

Example 25. A system comprising: a source of NO₂; a vessel comprising the media of any of the examples herein, particularly Examples 1-24; a patient interface coupled to the vessel and configured to deliver the nitric oxide to a patient.

Example 26. A method of making the media of any of the examples herein, particularly Examples 1-24, wherein the method comprises dry blending of the antioxidant material present in the solid form with the support material.

Example 27. The method of any of the examples herein, particularly Example 26, wherein the support material comprises a hydrated material.

Example 28. A method of making the media of any of the examples herein, particularly Examples 1-24, wherein the method comprises mixing a saturated or supersaturated solution of the antioxidant in a solvent with the support material; removing the solvent to form the antioxidant material present in the solid form.

What is claimed is:

1. A device comprising:
a media configured to convert nitrogen dioxide into nitric oxide, wherein the media comprises:
a) a support material; and
b) an antioxidant material, wherein at least 15 wt % of the antioxidant material is present in a solid form.

2. The device of claim 1, wherein the media further comprises a solvent present in an amount of 0 wt % to 40 wt % based on a total weight of the media.

3. The device of claim 2, wherein the solvent comprises water, an organic solvent, or a combination thereof.

4. The device of claim 1, wherein the antioxidant material is present in an amount greater than 0 wt % to less than 100% based on a total weight of the media.

5. The device of claim 1, wherein the antioxidant material present in the solid form is present in an amount greater than 50 wt %.

6. The device of claim 1, wherein the antioxidant material is present only in the solid form.

7. The device of claim 2, wherein when the solvent is present in an amount greater than 0 wt % to 40 wt %, the media further comprises a mixture of the antioxidant material present in the solid form and the antioxidant material dissolved in the solvent.

8. The device of claim 1, wherein the antioxidant material present in the solid form has a particle size of 1 to 1000 microns.

9. The device of claim 1, wherein the antioxidant material comprises ascorbic acid; alpha-, beta-, gamma-, or delta tocopherol; alpha-, beta-, gamma-, or delta-tocotrienol; polyphenols; beta-carotene; or a combination thereof.

10. The device of claim 9, wherein the support material comprises a molecular sieve, a metal-organic framework (MOF), natural zeolites, synthetic zeolites, polymers of intrinsic microporosity (PIMs), hyper-crosslinked microporous polymers (HCPs), covalent organic frameworks (COFs), conjugated microporous polymers (CMPs), porous aromatic frameworks (PAFs), porous organic cages (PCs), silica gel, or any combination thereof.

11. The device of claim 10, wherein the support material has a particle size of 1 to 1000 micrometers, and/or wherein the support material is a porous material has an average pore size of 1 to 1000 angstroms.

12. The device of claim 1, wherein the support material exhibits a specific surface area of 400 to 6000 m²/g.

13. The device of claim 1, wherein the media exhibits conversion efficiency greater than 60%, or greater than 80%, or greater than 90%.

14. The device of claim 1, wherein the device is capable of dosing up to 6400 ppm-LPM.

15. The device of claim 1, wherein the media is disposed within a vessel.

16. The device of claim 1, wherein the media is in fluid communication with a source of NO₂.

17. The device of claim 1, wherein the media comprises a fluidic pathway configured to transfer formed nitric oxide to a patient.

18. A system comprising:
a source of NO₂;
a vessel comprising the media of claim 1;
a patient interface coupled to the vessel and configured to deliver the nitric oxide to a patient.

19. A method of making the media of claim 1, wherein the method comprises dry blending of the antioxidant material present in the solid form with the support material.

20. A method of making the media of claim 1, wherein the method comprises
mixing a saturated or supersaturated solution of the antioxidant material in a solvent with the support material;
removing the solvent to form the antioxidant material present in the solid form.
